(19) United States
(12) Patent Application Publication
(10) Pub. No.: US 2026/0048275 A1
(43) Pub. Date: Feb. 19, 2026

(54) UV APPLICATION ENHANCEMENTS FOR IN OR ON-BODY PATHOGEN ERADICATION

(71) Applicants: Asher Baum, Brooklyn, NY (US); Israel Nissenbaum, Brooklyn, NY (US)

(72) Inventors: Asher Baum, Brooklyn, NY (US); Israel Nissenbaum, Brooklyn, NY (US)

(21) Appl. No.: 19/300,533

(22) Filed: Aug. 14, 2025

Related U.S. Application Data
(60) Provisional application No. 63/682,846, filed on Aug. 14, 2024.

(51) Int. Cl. A61N 5/06; A61B 1/00; A61B 1/12; A61B 10/04; A61L 2/00; A61L 2/10; A61L 2/24; A61M 1/00

(52) U.S. Cl. CPC A61N 5/0624; A61B 1/00142; A61B 1/126; A61B 10/04; A61L 2/0047; A61L 2/10; A61L 2/24; A61M 1/985; A61N 5/0601; A61L 2202/11; A61L 2202/14; A61L 2202/17; A61M 2205/053; A61N 2005/0626; A61N 2005/063; A61N 2005/0644; A61N 2005/0651; A61N 2005/0661; A61N 2005/0666

(57) ABSTRACT

An endoscope having a UV light emitting LED on a distal end of its insertion tube for pathogen eradication within a body or object or a UV emitting light source in the handle of the endoscope optically connected to a transmission fiber extending to the distal end. Devices for maintaining uniform power of UV light on a pathogen containing site of either a spacer for direct constant contact or a distance sensor which correspondingly changes output power for uniform power impingement. A bandage-like device for disinfection having UV transmitting fibers or UV emitting LEDs placed in close proximity to a wound or infected body site, for effecting disinfection and maintaining sterility against infective pathogens. A device with confined heat generating UV light emitting LED having a pulse generator for reducing heat while maintaining pathogen eradication. A hollow core fiber optically connected to a UV pulse laser which is resistant to degradation at the connection.

### FIELD OF THE INVENTION

This invention relates to expedients for enhancing use of UVC light (defined as being the wave-length region of between 200 to 280 nm and particularly 222 nm for safety and 265 nm for pathogen eradication effectiveness) with the DNA/RNA disruption and eradication of pathogens in vivo in or on human and animal bodies.

### BACKGROUND

In various patents (e.g. U.S. Pat. No. 11,554,187) and pending patent applications, Applicants herein have disclosed and provided various devices and methods for the transmission of UVC light for the treatment of humans and animals and the sterilization of devices, and specifically medical devices, with the eradication of pathogens such as viruses and bacteria and including cancer (cancer cells, though not technically pathogens are broadly defined, for purposes of this and prior patents and applications cancer as pathogens (i.e., harmful or potentially harmful and having DNA/RNA). Also included in the term “pathogens” herein are any biological entities having DNA/RNA subject to disruption and which are harmful or potentially harmful.

UV pathogen impingement power output (and particularly UVC) has been severely limited, especially in constricted dimensions of body parts such as ducts and small channels, which are difficult to enter and reach. Similarly, confined areas of medical instruments and other devices subject to infection with pathogens are also severely limited. In addition, small devices and transmission elements containing and/or used for transmitting UVC light have also been limited for practical use by factors of heat generation and heat damage, with necessary and often difficult to provide heat sinking. In addition. There is the damage to small device and transmission elements, particularly of polymeric materials, by the highly corrosive UV light. In addition, there is the high tendency of UVC light, particularly from direct light sources such as LEDs and various bulbs, to scatter and thereby frustratingly dissipate pathogen eradication power, when needed to be precisely directed.

UV light emitting LEDs (particularly in the UVC range of about 200 to 280 nm) have particularly low efficiencies, generally of about 1-2% (e.g., an electrical input of 1 watt provides, generally at most, with a 10 to 20 milliwatt UV light output though sometimes up to 50 milliwatts) with very high amounts of heat being locally generated by the unused input power. Prior attempts at using the pathogen disruption power of UV light, involving placement of UV LEDs directly in the body, have generally been rendered difficult or effectively unusable because of harmful heat generation. Methods and structures have been devised to effect heat dissipation or heat sinking (see e.g., Gertner—US Patent Publication 2006/0195165) but have been generally ineffective as a practical matter.

UV emitting lasers, particularly those using pulse emissions, embody an additional utilization problem, in conjunction with coupling to small diameter fibers for internal body insertion use. Normal pulse rates, even if of very small time durations, may catastrophically burn the fiber at its connection with the laser, rendering it almost initially inoperable.

### SUMMARY

It is accordingly an object to provide procedures, such as “surgical” methods with minimized dimensional needs for UV and particularly UVC light sources such as LEDs, while maintaining sufficient pathogen eradication power and effectively minimizing heat generation detrimental effect. As used herein, the term “UV” encompasses DNA/RNA disrupting wavelength in the UV range spectrum of between 200 nm to 340 nm and particularly the UVC wavelength range of between 200 nm to 280 nm, with maximum DNA/RNA disruption capability and minimal tissue penetration (generally less than 40 microns).

It is a further object to configure UV and particularly UVC carrying devices such as endoscopes, catheters and the like to directly and effectively bring or position generated UV or UVC light from a UV or UVC light source to pathogens within the body or to maintain a sterile environment, free of pathogens, at operative surgical or prophylactic treatment sites within or on a human or animal body.

It is another object of the invention to provide heat degradation resistant fibers such as hollow core fibers for the transmission of UV and particularly UVC light, particularly with pulsed UV and particularly UVC lasers, with little or no damaging heat degradation of the fiber.

It is yet another object to provide a UV and particularly UVC LED pulsed light generation system which maintains pathogen eradication power while minimizing generated heat, particularly for hand-held devices.

In another object, the wave-length of the light from the UV light emitting source is within a range wherein the light has no effective tissue penetration (i.e., between 200-230 nm) whereby the use thereof is undeniably safe.

It is still another object to provide a universal wound sterilization or disinfection “bandage” or adherent device, applied as a wound covering, particularly for military in-field applications, which is effective, regardless of infection source, with low power direct UV light for extended time periods, without or with minimal detrimental heat generation or the need to identify infection causing pathogens. The bandage or wound covering adherent device further encompasses long term (hours or days) continued sterilization or disinfection of external body parts subject to infection damage other than wounds such as diabetic lesions.

The above and other objects, features and advantages of the invention will become more evident from the following discussion and drawings in which:

### SHORT DESCRIPTION OF THE DRAWINGS

FIG. 1 shows the distal insertion end of an endoscope modified with the inclusion of a sterilizing small UVC light-emitting LED;

FIG. 1A depicts the LED of FIG. 1, being provided with a TIR lens (the lens is shown enlarged for clarity) for UVC power extension;

FIG. 1B shows the LED of FIG. 1, provided with a collimating lens (the lens is shown enlarged for clarity) for the UVC power extension;

FIG. 2 shows a small UVC LED positioned at the distal end of the biopsy channel of an endoscope;

FIG. 2A is a schematic cross section view of an endoscope with a UVC emitting LED positioned within the handle thereof and with alternative transmission-elements carrying UVC light passing through the biopsy channel or through a separate channel formed in or on the endoscope;

FIG. 2B is a view of an optional transmission-element structure having a distal brush element;

FIG. 3 shows an endoscope with a standard insertion-assisting outer sleeve, with a small UVC LED and its power input being supported in position within the sleeve;

FIG. 4 depicts a spacer element fitted over the end of the endoscopes of FIGS. 1-3 which is open to transmission of UVC light emitted by the LED and resistant to degradation by the UVC light;

FIG. 5 shows the endoscopes of FIGS. 1-3 providing a sterilizing surgical environment within an exemplary internal surgical area of a stomach, susceptible to introduction of infecting pathogens;

FIG. 6 is a partial schematic cross section view of a hand-held UVC light emitting device with a pulsed output and a reduced heat generation in a confined area;

FIG. 7 is a schematic cross section view of a hollow core fiber suitable for use with pulsed UVC lasers with minimized laser peak power damage and a table of its parameters;

FIG. 8A is a perspective view of a disinfecting “bandage” type adhering device suitable for military injury use with a wound covering area comprised of laterally UVC emitting fibers in a wound contacting or covering gauze “area”, with integrated power supply and UV light source;

FIG. 8B is a perspective view of the “bandage” type adhering device of FIG. 8a with separate power unit and light source module;

FIG. 9 is a view of the disinfection bandage-type adhering devices of FIGS. 8a and 8b with low power emitting UVC LEDs in a minimal heat generating configuration structure forming the wound (or infected site) contacting or covering “gauze” area; and

FIG. 10 shows a “WoundVac” machine cleaning a wound prior to deployment of the disinfecting bandage type adhering devices of FIGS. 8a, 8b and 9.

### SUMMARY

Generally, embodiments of the invention comprise the enhanced configuration and utilization of UV LEDs in effecting sterilization and pathogen eradication within or on human or animal bodies. Most effectively, the UV light is in the UVC range and reference herein is to the UVC light which encompasses the most effective DNA/RNA disruption with the least amount of tissue penetration. The vast majority of bacteria and virus pathogens are surface situated thereby serendipitously providing the most disinfection with limited risk to healthy tissue.

In several embodiments, the LEDs are brought more directly in proximity with sites requiring sterilization to maintain a sterile operating procedure environment and or pathogen eradication to remove existing pathogens or pathogens introduced, as a result of the procedure. Because UV emitting LEDs are of relatively large dimensions, they were not initially capable of being directly introduced into a human or animal body through many highly dimensionally restricted bodily ducts. In particular, relatively high-power (i.e., preferred in order to safely and reliably eradicate pathogens) UV LEDs have become increasing larger and less amenable to body insertion because of constricted insertion and operative area dimensions, as well as detrimentally increased heat generation. In addition, effective optical coupling to transmission media (as disclosed in applicants' U.S. Pat. No. 11,554,187), with effective pathogen eradication power, has been rendered more difficult. Larger size emitting diode dies (the UV generating area of the LEDs) are inherently incompatible, with efficient light transmission through small diameter transmission media such as optical fibers suitable for insertion into body ducts and passages with mismatches in size (etendue light limitations of light power not normally being effectively compressible) and loss of significant power.

However, recently, relatively low power UV LEDs have been developed with smaller dimensions and lower heat generation, actually rendering them capable of being safely inserted and moveable within various body ducts and passages, though with relatively low emitted power. In accordance with embodiments herein, the small LEDs are able to provide greater direct-site UV power impingement on pathogens than larger high-power LEDs. The high-power LEDs have entailed significant transmission power losses, prior to any effective direct-site power application.

Generally, a first embodiment comprises a device capable of providing pathogen eradication and disinfection within a human or animal body or a pathogen containing object with restricted entry therein. The pathogen is defined herein as comprising any of viruses and bacteria and cancer and any biological entities having DNA/RNA subject to disruption and which are harmful or potentially harmful. The device is comprised of an insertion tube of appropriate dimensions for the body or object insertion, having proximal and distal ends. The distal end comprises an opening for a channel for insertion of operative devices into the body or object and an illumination port for illuminating an interior portion of the body or object. An LED is positioned on the distal end which is configured to emit UV light in the range of 200 nm to 340 nm onto pathogens in the illuminated interior portions, with sufficient power to effect substantial pathogen eradication and disinfection within the illuminated body portions or object interior. An endoscope, defined as an instrument used for insertion into animate bodies and inanimate objects for interior evaluations and/or operative functions therewithin, is an example of the device.

In another embodiment the device is an endoscope wherein the distal end of the endoscope has an insertion end “real estate” area taken up by a biopsy channel opening as well as openings for water and air emission, as well as a light source and camera for the viewing endoscopic function. However, a small area remains which is sufficient for the implanting of a small UVC LED (e.g., a UVC LED with an 0.9 mm diagonal having a 20 mW power output is currently commercially available). In such embodiment, a powering wire is imbedded in the polymeric endoscopic material for connection to an outside power source, similar to the arrangement which powers the imbedded camera. With the imbedding, the endoscopic material provides more than adequate heat sinking for the LED. To enhance the utilizable emitted power to a site (with UVC power being substantially maintained for an extending distance), the UVC LED is provided with a small collimating lens, reflector or lens equivalent or UVC light transmitting element fitted over the end of the endoscope. The type of lens also encompasses any lens such as a TIR lens (if possible to be made of utilizable size), made suitable for UVC light transmission, which is capable of “re-imaging” or extending a UVC spot with pathogen disrupting power, at a distance from the LED, to compensate for distancing of the lens from a pathogen site.

For maintaining a fixed distance between LED and irradiated surface with substantially uniform power emission, a UVC light transmitting spacing element or open spacer is affixed to the distal end of the endoscope. With such spacer, the endoscope end, with contained UVC LED, is placed on and moved directly above a surface with a constant small spacer distance and direct UVC pathogen eradication power being consistently and reliably applied to a pathogen site with a known pathogenic eradication effect. In a less preferred structure, though included in the invention herein, the spacer element is substantially solid but is transmissive and degradation resistant to UVC light passing therethrough. It is understood that reference to UVC herein encompasses the general UV DNA/RNA disruption range with the UVC spectrum being the most efficient for disruption, with the least tissue penetration. In a further embodiment, the spacer element is flared or angled outwardly with the interior surface being made reflective such as with a metallic coating. The flared angle is such that UV light, with pathogen eradication power emanating from the distal end of the endoscope, which is angled away from a target pathogen site and normally lost from use, is reflectively diverted toward the target pathogen site.

Alternatively, a feedback distance measuring sensor or device, such as LiDAR, between emitted UV light and target surface, is used to maintain a relatively constant or controlled power application to the target surface. The distance between the UV light emission (distal end of fiber or the LED where used without a fiber) and the target surface is constantly monitored during use and with distance changes, the power output is varied such that the power actually impinging on the target surface remains substantially constant, such as in terms of milliwatts per square cm. (e.g., closer distances result in lower power output and further distances result in increased the power output, all on a basis of the inverse square rule), to maintain a constant surface impingement of UV light per designated area units.

In another embodiment, a sheath or sleeve that is normally utilized with the insertion tubing of an endoscope, to facilitate body insertion, is used to enclose and hold a small UV LED and its wiring. In such embodiment, the LED is held at the distal end of the sheath and enclosed therewithin against the outer surface of the endoscope insertion tube, with the LED positioned to face away from the endoscope, toward pathogen sites in proximity to the endoscope end. The adjacent insertion tube provides requisite heat sinking, with the sheath or sleeve preventing detrimental external heat dissipation within the body.

In embodiments utilizing the biopsy channels of endoscopes, a UV LED of a size sufficient to enable it to be placed into the endoscope is positioned at the distal end of the biopsy channel with the power supply wire being drawn through the biopsy channel or alternatively through a separate duct formed in the polymer of the endoscope. In reverse embodiments, the UV LEDs are imbedded in the handle of the endoscope and a transmitting fiber either extending through the biopsy channel or through a separate duct formed in the polymer of the endoscope. In all embodiments where UV LED light is directly trained on target pathogens, the UVC LED is optionally provided with collimating or other lenses (e.g. TIR or UV spot distancing lenses with maintained UV power) depending on the intended utilization, to enhance UV pathogen eradication effect. As an optional feature, the transmitting fiber includes a physical disruption distal element, such as a brush, which serves to dislodge physical debris which may impede UV light directly impinging on sites. The brush may be integrated with the fiber or be removable therefrom for periodic replacement. The brush is sized to permit ready insertion in tubing or channels requiring disinfection with the bristles folding to forcibly dislodge the physical debris prior to impingement of the disinfecting UV light on exposed pathogens. It is understood that all transmission components, including fibers and lenses are UV transmissive and generally resistant to UV degradation (unless for single or limited use), with other components subject to UV light impingement are similarly UV resistant. In some embodiments, the brush is sized and configured to provide a sloughing action on UV applied sites, to remove dead cells between UV site applications to effectively permit deeper UV penetration where necessary or desired.

The wave-length of the respective UV LEDs for pathogen eradication resulting from DNA/RNA disruption is in the UV range of 200 to 340 nm with the wave length range of 200-230 nm (particularly 222 nm) without any penetration characteristics and where depth of penetration is not wanted, with safe use being paramount, such as in sensitive areas such as in the eyes. In the range of 250 nm-280 nm and particularly at 265 nm, pathogen eradication is most effective and rapid but with a slight depth of penetration of about 40 microns. Such slight penetrating wavelength is useful where small amounts of healthy cells may be affected in exchange for rapid effectiveness and where cell regrowth is expected. Though operating at lesser effectiveness in pathogen eradication, wave lengths in the range of 280 nm to 340 nm provide greater depth of penetration where required for such purpose. LEDs with UVC light wavelength of 285 nm (just outside the UVC range) have recently, while nominally providing less DNA/RNA disruption power than the 265 nm LEDs, compensated for the reduced inherent pathogen disruption by having greater emitted power output (and overall greater pathogenic eradication effect) particularly in currently available smaller dimensioned LEDs.

A direct benefit of the embodiments of UV LED placement in direct line of sight with the pathogens within a body is that of site sterilization within the body, particularly in areas wherein surgical or other invasive procedure may result in the introduction of infectious materials. The small LEDs may be used in initial steps or with constant or controlled UV light generation to insure maintenance of a sterile environment. Where surface sterility is required, lower wave-length non-penetrating UV light such as at 222 nm may be deployed with substantial safety and reduced concern regarding effect on healthy cells.

Heat generation, particularly with internally utilized UV LEDs or within hand-held devices often becomes a problem with concomitant tissue damage or high heat in a handling device and possible heat damage to the LEDs in confined spaces. Accordingly, in other embodiments where heat generation is problematic and wherein heat sinking is not feasible or practical, a pulse generator is provided in conjunction with a power supply, for powering the respective LEDs. With such expedient, heat generation is drastically reduced to tolerable levels while peak power pulses are provided for effective pathogen disruption. UV emission within pulsed times are such that the biologically targeted pathogens are unable to recover or resist the UV pathogenic disruption. Peak intervals and pulse power output are adjusted to reduce generated heat to tolerable levels (generally up to about 40-45 degrees C. but without limitation thereto) while maintaining average effective output power.

In U.S. Pat. No. 11,554,187, a UV light source is disclosed as being coupled to a transmission medium for the effective transmission of UV light to pathogen containing sites for the eradication of pathogens. One of the examples of a transmission medium is that of a fiber optic cable. Consideration for suitable fiber optic cables is that it be comprised of a material that is resistant to UV degradation for at least a period of useful utilization.

In a secondary consideration, if the UV light source is a laser, the most common laser is that of a pulsed configuration. However, pulsed lasers, when used with smaller diameter fibers (generally less than 200 microns), are limited in output, since output in the milliwatt range entails pulsed energy peaks of kilowatts. The small diameter fibers, particularly at initial connection sites, prior to power dissipation, are not able to sustain integrity without catastrophic disintegration, even though the pulses are of very short duration.

Accordingly, in an embodiment, with particular use of UV light emitting lasers, and particularly with pulsed lasers, optical fibers of the class known as hollow core fibers are useful in resisting fiber damage at laser coupling sites, most particularly with small diameter fibers (e.g., with diameters of 200 microns or less) and when used with pulsed UV lasers having high energy peak pulses wherein concentrated energy in the small diameter is normally susceptible to fiber damage. Hollow core fibers, with a solid containment sleeve, are often configured with contained air pockets which provide a natural heat sink for built up heat and wherein UV light is transmitted therethrough by reflective bouncing off mirrored surfaces lining the interior of the fiber. In an alternative embodiment, a glass section is used to separate the laser input from the fiber (generally of specially treated UV resistant quartz silica) at the connection in order to dissipate the short duration pulse power from directly contacting the fiber material.

There is a current need, particularly in battle area applications, for wound bandages, typically used to prevent hemorrhaging (commonly known as Israeli bandages), to be followed up with separate wound or skin coverings with bandage-like wrapping, adhesive or other adhesions, i.e., adhesion devices, generally after hemorrhaging has been controlled, for providing continued disinfection or sterilization against dangerous infections and sepsis in the wound, for periods of hours and possibly days. However, because of the disparate nature of infecting pathogens it has been necessary to initially identify the type of infecting pathogen, in order for a disinfection bandage to be properly provided which has appropriate disinfecting agents. In some cases, no readily available disinfectant is effective against some pathogens.

Accordingly, in an embodiment herein, an adhesion device with an adhesive or other adhering means comprising a supporting element similar to a bandage, is provided with or in place of the gauze of the normal bandage (typically sized to contact or cover a wound), being replaced or provided with interwoven or more commonly matted (side by side) fibers optically attached to a UV light source, most typically, one or more UV LEDs. For patient mobility, a mini power supply or battery, attached to the UV light source (e.g., LEDs) is positioned on the adhesion device, obverse to the fibers, with the UV light source being optically attached to the fibers, through the adhesive supporting element (connection is on either side of the adhesive supporting element). Alternatively, such as for a non-mobile patient, a separate unit of either or both of a power supply and UV light source are attached to the fibers to power and provide sterilizing UV light to the wound on a continuous or controlled basis.

The fibers are configured to have UV light emitting sections, whereby UV light passing through them is directed toward the wound to effect continuous (or timed) DNA/RNA disruption of any and all infecting pathogens in the wound, thereby effectively disinfecting or continuously sterilizing the wound, regardless of type of pathogen (all biological pathogens have DNA/RNA). Since placement of the bandage is not momentary but for a significant period of time, effective UV power output for disinfection is cumulative over time and the continuous constant power output can be relatively low (e.g. ranging from several hundred microwatts or even less to about one or two milliwatts or even more from either a low power LED or a higher power LED with regulatable output). Actual power output levels (according to bandage type and use) are either adjustable or separately available according to wound type and dimensional extent as well as application time. Small battery units with minimized heat generation and the powered LEDs are positioned to be directed away from the wound, with small power output further serving to prevent detrimental heating of the wound. Ideally, the power supplies are replaceable and/or rechargeable and the LEDs and fibers units are recyclable for additional utilizations when the continued use for a particular application has ended. The battery power supply, LEDs and fiber structure are removable from the supporting adhesion device for recycling purposes and the supporting adhesion device may be discarded. Other similar applications include sterilization of open sores, cankers, and lesions commonly occurring in the legs or limbs of diabetics and for the general disinfection treatment of skin diseases such as psoriasis.

In an alternate embodiment, small output LEDs (generally of 10 milliwatts or less or controlled higher powered LEDs) are directly placed in the gauze area of the adhesion device with a UV LED output directed and, optionally facilitated with lens elements, into a wound. The UV LEDs are, in one embodiment, encapsulated with liquid (e.g., blood and other bodily fluids) protective materials (e.g., with UV transmissive materials such as FEP, PTFE and EPTFE films, PFA and the like) which are permeable by or allow transmission therethrough of UV light and which are resistant to UV degradation. Wound size is determinative of the number of LEDs necessary for placement to effect sufficient disinfection and/or wound sterilization. Matching of appropriate size adhesion devices to wound sizes facilitates full wound disinfection.

### DETAILED DESCRIPTION OF THE DRAWINGS

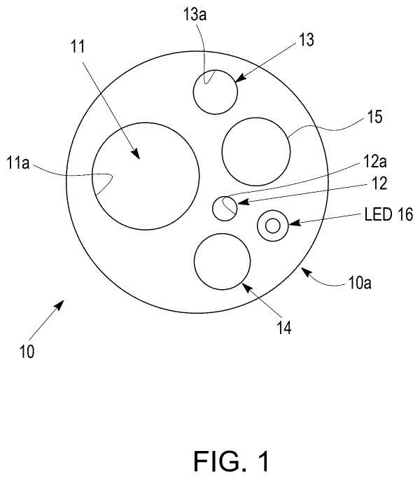

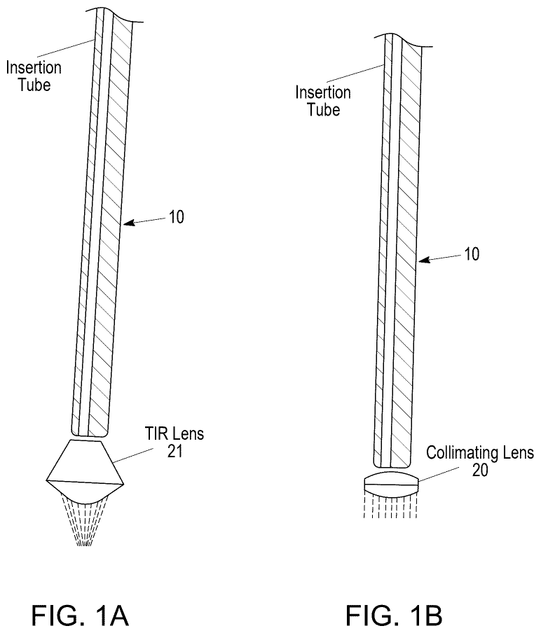

FIG. 1 shows the distal end 10a of a typical endoscope 10 with openings 11a, 12a, 13a for biopsy channel 11 and water and air introduction channels 12 and 13 respectively. A light emitting fiber 14 provides illumination which is captured by and relayed by a camera element 15 to the viewing operator which enables proper directing of the UVC light on pathogenic or possible pathogenic sites. A small UVC emitting LED 16 is imbedded in the end of the endoscope, between the various channels, with powering wire (not shown) being imbedded in the polymer of the endoscope for external power connection. UVC light from the LED 16 is controllably trained on sites for maintaining sterilization thereof or for pathogen eradication treatment. An optional lens, such as a collimating lens 20 (FIG. 1B), facilitates training and effectiveness of UVC at a distance from a pathogen containing or sterilization site within the body. Another examples of a lens utilizable for extension of the power effect of the LED 16 is a TIR lens 21 shown in FIG. 1A. Other lenses, reflectors and the like with appropriate power extension or utility for pathogen eradication may be utilized as well. It is noted that lenses are nearly always used with visible light to extend viewing capability. UVC light in the pathogenic disruption range is essentially not visible and the primary purpose of the lens is for effective “extension” of power capability of the UVC light.

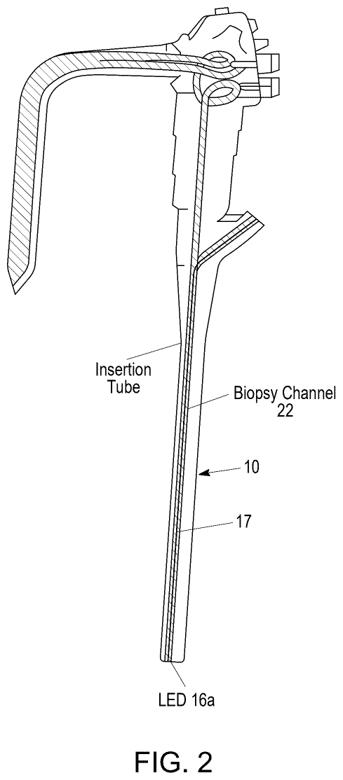

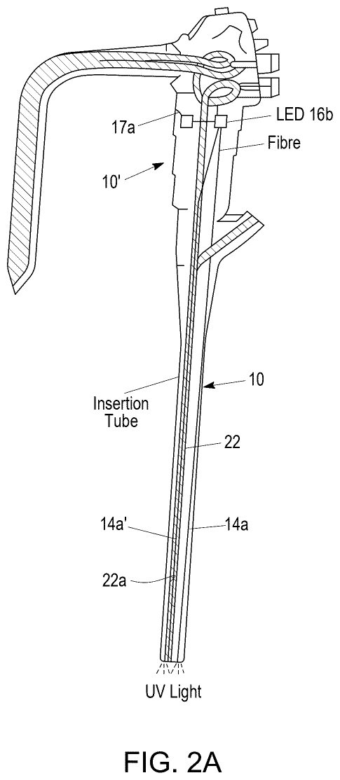

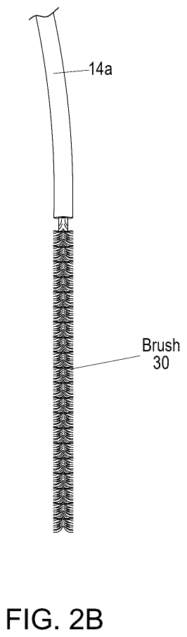

FIGS. 2 and 2A show various effective sites for placement of a UVC emitting LED 16a, at the end of the biopsy channel 22 (FIG. 2) by essentially passing the UV LED power line 17 through the biopsy channel 22 and fitting the LED 16a at the distal end of the biopsy channel 22. Alternatively, as shown in FIG. 2A, the LED 16b is imbedded with power linkage 17a into the handle 10′ of the endoscope 10 with a UV transmitting fiber 14a passing through the biopsy channel 22 or separately configured to pass through the solid polymer of the endoscope 10 (with both shown as alternatives).

In FIG. 2B a fiber 14a, used for passing through the biopsy channel 22 such as shown in FIG. 2A, is initially provided with a preferably removable distal end brush 30 configured to disrupt physical debris in the channel 22 prior to UVC light impingement to facilitate direct UVC light impingement on the interior walls 22a of the biopsy channel 22 or sterilization of the biopsy channel 22 by dislodgement or removal of debris which may block the sterilization effect of the UVC light.

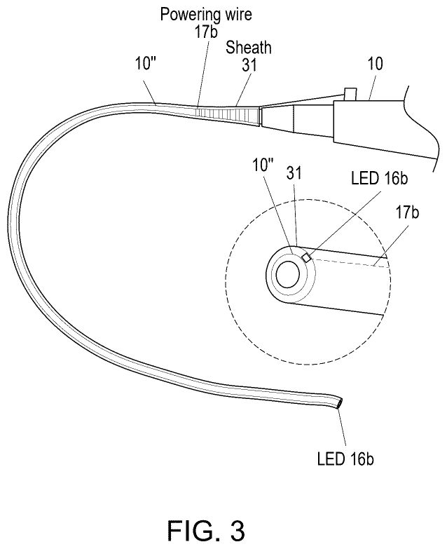

FIG. 3 shows an alternative, removable placement of a UVC LED 16 and its powering wire 17b between a sleeve or sheath 31 closely fitted on the insertion tube 10″ of the endoscope 10, with the LED 16 positioned at the distal end of the sleeve 31 and insertion tube 10″ to provide a forward and/or radial emission of UVC light to a sterilization or pathogen eradication site.

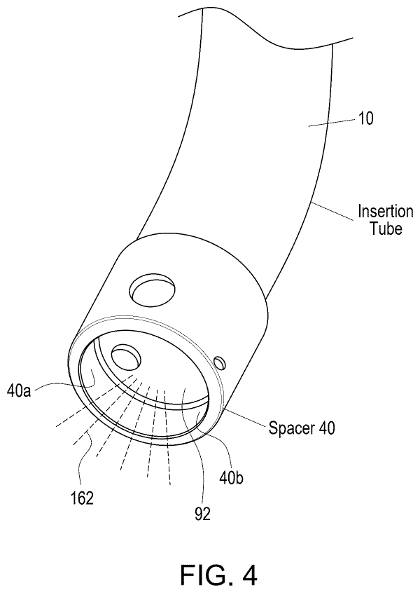

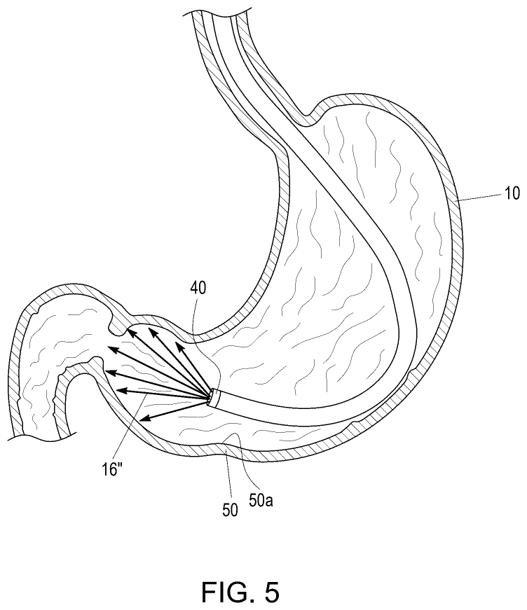

FIG. 4 depicts the ends of the endoscopes 10 of FIGS. 1-3 being fitted and covered with a small spacer element 40 which is transmissive of UVC light from the LED 16 of the endoscope 10 and resistant to degradation thereof. The spacer element 40 is configured to directly contact the surface of an internal lumen 50 wherein constant or controlled power UVC light impinges on surfaces of the lumen 50 with the distal end of the endoscope 10 with emitted UVC being moved directly along the lumen surface 50a with substantially constant and uniform UV power output. Alternatively, the distance from the UVC light output at 162 and lumen surface 50a (FIG. 5) is constantly monitored by LiDar sensor 92 which is connected to the controls (not shown) of LED 16 to concomitantly vary output UVC power such that UVC light 16″ impinging on lumen surface 50a remains constant. The walls 40a of space 40 are slightly flared and angled with the inner surface 40b being coated with a reflective coating whereby light output 162 which is directed outward is reflected by coated surface 40b toward the open end of the spacer 40.

FIG. 5 schematically depicts the sterilizing surgical environment as effected by the endoscopes 10 of FIGS. 1-4 on an internal surface of a stomach 50a as an example of an internal surgical site, whereby the impinging (and movable if necessary) UVC light 16″ provides a sterilization effect to prevent infection and possibly sepsis as a result of a surgical or other similar medically mandated procedure.

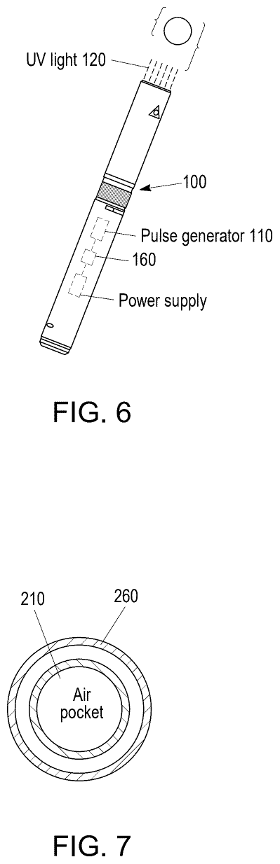

In FIG. 6, a hand-held device 100 of small dimension, with contained UVC emitting LED 160, is provided with a pulse generator 110 which controls UVC light output 120 to be short duration pulses, with minimized heat generation but with sufficient impingement power on the DNA/RNA containing target pathogens to maintain biological eradication effect, without pulse recovery.

FIG. 7 is a schematic cross section view of a hollow core fiber 260 showing an enclosed air pocket 210 and reflective UVC light transmission with sufficient heat dissipation to render it less susceptible to pulse power damage by the pulse UVC laser.

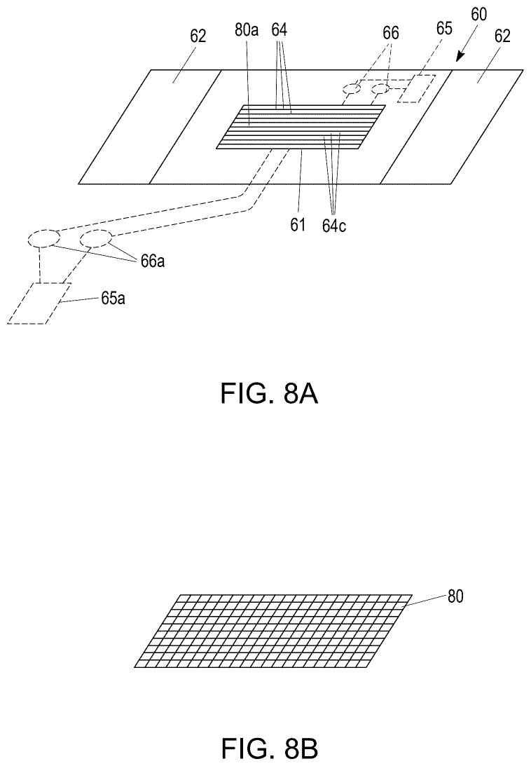

FIG. 8A is a perspective view of a sterilization adhesion device 60 having adhesive areas 62 for fixed placement of the device on a wound and with a UVC emitting sterilization section 61 comprised of sterilizing light emitting fibers 64. A power supply battery 65 is on the obverse side of the adhesion device 60 together with one or more UVC LEDs 66, typically of low power to minimize heat output and for long term low power sterilizing UVC output. The fibers 64 are optically connected to the UVC LEDs 66 through the device 60. The fibers 64 with attached UVC LEDs 66 are separably removable from the device 60 for re-use. The fibers 64 are arranged in an interwoven array 80 or a matted arrangement 80a in FIG. 8B, wherein, in both arrangements, the sections of the fibers 64a facing the wound are rendered laterally transmissive of the UVC light whereby the UVC is able to cumulatively impinge on the wound or possible infection site for sterilization thereof. The device 60 with integral power supply battery 65 and UVC LEDs 66 is primarily for a mobile patient. As shown in dotted lines, either or both the battery or other power supply 65a and UVC LEDs 66a (or other UVC light source) may be separated from the adhesion device 60, for non-mobile patients

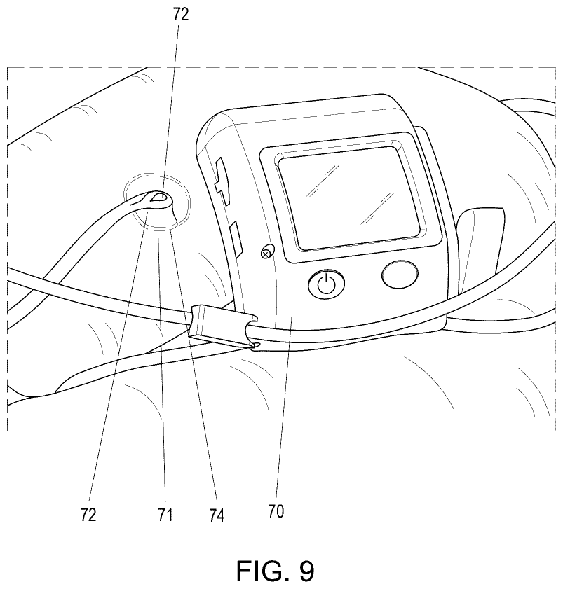

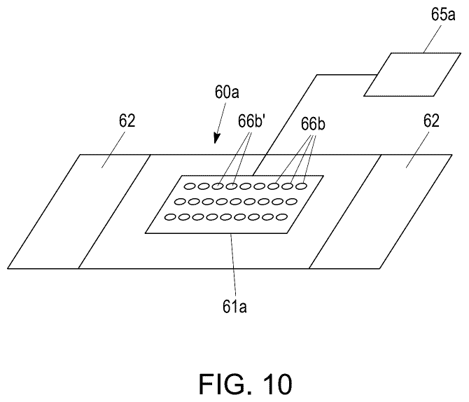

FIG. 9 shows a wound clearing machine (WoundVac) 70 being used to clear detritus from a wound or possible infection site 71, prior to application of the bandage of FIGS. 8A and 8B to the possible infection site. The “vacuum area” 72 is attached to the skin around a wound 74 and removed when the clearing is complete. The sterilization device 60 of FIGS. 8 and 60a of FIG. 10 is thereafter placed on the wound for continued sterilization.

FIG. 10 shows an alternative sterilization adhesion device 60a with small, relatively low power UV LEDs 66b being directly positioned in a sterilization section 61a, with the UV light emitting dies 66b′ of the LEDs 66a facing a wound to which the sterilization device 60a is applied.

It is understood that the drawings and descriptions including numerical parameters contained therein are illustrative of the invention and that changes may be made in the structure and configuration and with components with illustrative parameter values and ranges without departing from the scope of the invention as defined in the following claims.

What is claimed is:

1. A device capable of providing pathogen eradication and disinfection within a human or animal body or interior of a pathogen containing object, wherein the pathogen comprises any of viruses and bacteria and cancer and any biological entities having DNA/RNA subject to disruption and which are harmful or potentially harmful, the device being comprised of an insertion tube of appropriate dimensions for the body or object insertion, with proximal and distal ends and wherein the distal end comprises an opening for a channel for insertion of operating devices into the body object, and an illumination light port for illuminating interior portions of the body, characterized in that an LED is positioned on the distal end and configured to emit UV light in the range of 200 nm to 340 nm onto pathogens in the illuminated interior portions of the body or object with sufficient power to effect substantial pathogen eradication and disinfection within the illuminated body portions or object interior.

2. The device of claim 1, wherein the device comprises an endoscope for use in a human or animal body and the channel is a biopsy channel and wherein the LED is protected from degradation by body fluids by a UV transmissive material and wherein a lens element is positioned adjacent UV light emitted by the LED and configured to effectively extend the distance of the pathogen eradication from the LED.

3. The device of claim 2, wherein the lens element comprises at least one of a TIR lens and a collimating lens.

4. The device of claim 2, wherein the LED is positioned within the opening of the biopsy channel.

5. The device of claim 1, wherein the insertion tube is contained within a closely fitted sleeve and wherein the LED is positioned at the distal end and held between the sleeve and an outer wall of the insertion tube.

6. An endoscope capable of providing pathogen eradication and disinfection within a human or animal body wherein the pathogen comprises any of viruses and bacteria and cancer and any biological entities having DNA/RNA subject to disruption and which are harmful or potentially harmful, the endoscope being comprised of an operative handle portion connected to body insertion tube with proximal and distal ends and wherein the distal end comprises an opening for a biopsy channel, an illumination light port for illuminating interior portions of the body, a camera port for capturing and sending images of the illuminated body portions to an external monitor characterized in that a UV light source is positioned in the handle portion and optically connected to a UV transmission element extending through the biopsy channel or through the body insertion tube to the distal end, the UV light source being configured to emit UV light in the range of 200 nm to 340 nm through the distal end of the transmission element onto pathogens in the illuminated interior portions with sufficient power to effect substantial pathogen eradication and disinfection within the illuminated body portions.

7. The endoscope of claim 6 wherein the transmission element comprises a brush member positioned at a distal end of the transmission element wherein the brush member is configured to effect any one of physical cleaning within the biopsy channel, cleaning of the interior body portions and removal of dead cells to enhance pathogen eradication and disinfection of the UV light on the cleaned areas.

8. The device of claim 1, wherein the distal end is provided with an open cylindrical spacer whereby the distal end is spaced from the body portion by a fixed distance when the spacer directly rests on the body portion whereby a fixed amount of UV light power is emitted onto the pathogens.

9. The device of claim 8, wherein lateral walls of the spacer are angularly flared outwardly away from the distal end and wherein an interior of the flared walls is reflective whereby they redirect laterally scattered UV light from the distal end to be substantially in line with the distal end and pathogens targeted for eradication.

10. The device of claim 1, wherein the device is an endoscope and further comprises a constant distance sensor between the UV LED and the body portion and wherein the sensor is configured to vary the power of UV light being emitted from the UV LED to maintain a substantially constant UV eradication power on the pathogens.

11. The endoscope of claim 6, wherein the distal end is provided with a spacer whereby the distal end is spaced from the body portion by a fixed distance when the spacer directly rests on the body position whereby a fixed amount of UV light power is emitted onto the pathogens.

12. The endoscope of claim 6, wherein the endoscope comprises a constant distance sensor between the distal end of the transmission element and the body portion and wherein the sensor is configured to vary the power of UV light being emitted from the UV light source to maintain a substantially constant UV eradication power on the pathogens.

13. A sterilizing adhesion device comprising an adhesion support element having an upper and a lower side, with the lower side configured to be placed on and adhered to a surface of a body part or object, having infectious pathogens thereon, for extended periods of time for effecting and maintaining sterilization thereof, the device further comprising a UV light source capable of emitting UV light in the range of 200 nm to 340 nm optically coupled to at least one UV light transmission element positioned on the lower side of the adhesion support element at a positioned directly adjacent to the infectious pathogens, when the adhesion support element is positioned on the surface of the body part or object, wherein the UV light transmission elements are configured to transmit UV light onto the infectious pathogens from the UV light source with sufficient UV light power during the time the adhesion device is on the body part or object to effect and maintain sterility with removal of the infectious pathogens.

14. The sterilizing adhesion device of claim 13 wherein UV light source and a power source therefor are positioned on the upper side of the adhesion support element, with the optical coupling between the UV light source and transmission elements extending through or around the adhesion support element, wherein heat from the UV light source is directed away from the body part or object.

15. The sterilizing adhesion device of claim 13 in combination with a woundvac device wherein the adhesion device is configured for placement on a wound which is cleaned from detritus prior to placement of the device on the wound whereby emitted UV light is able to directly impinge on the infectious pathogens.

16. A sterilizing adhesion device comprising an adhesion support element having an upper and a lower side, with the lower side configured to be placed on and adhered to a surface of a body part or object, having infectious pathogens thereon, for extended periods of time for effecting and maintaining sterilization thereof, the device further comprising a UV light source capable of emitting UV light in the range of 200 nm to 340 nm positioned on the lower side of the adhesion support element at a positioned directly adjacent to the infectious pathogens, when the adhesion support element is positioned on the surface of the body part or object, wherein the UV light source is configured to transmit UV light onto the infectious pathogens with sufficient UV light power during the time the adhesion device is on the body part or object to effect and maintain sterility with removal of the infectious pathogens.

17. The sterilizing device of claim 16, wherein the UV light source comprises multiple UV LEDs which emit low UV light power sufficient to effect and maintain sterility during the time the adhesion device is on the body part or object to effect and maintain sterility with removal of the infectious pathogens without significant harmful heat generation.

18. A device for pathogen eradication and sterilization comprised of a UV light source comprising at least one UV light emitting diode contained in a confined area whereby UV light emission from the diode is at a power level sufficient for the eradication of pathogens at which it is directed, wherein a harmful level of normally generated heat is ameliorated with the inclusion within the device of a pulse generator for the UV light from the at least one LED whereby low power levels provide UV light pulses sufficient to eradicate the pathogens at lower levels of heat generation.

19. A laser device configured to emit pulsed UV light, optically connected to a UV transmission fiber for transmission of UV light for pathogen eradication, wherein heat generated in UV light pulses is absorbed prior to reaching the optical connection by a UV transmissive and absorbent member or directed away from the optical connection by a hollow core transmission fiber.
